(19) United States
(12) Patent Application Publication
(10) Pub. No.: US 2009/0125005 A1
(43) Pub. Date: May 14, 2009

(54) Closed Loop System and Method for Ablating Lenses with Aberrations

(75) Inventors: Dimitri Chernyak, Sunnyvale, CA (US); Charles Campbell, Berkeley, CA (US); Jeffrey J. Persoff, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AMO Manufacturing USA, LLC, Santa Clara, CA (US)

(21) Appl. No.: 12/353,842

(22) Filed: Jan. 14, 2009

Related U.S. Application Data
(63) Continuation of application No. 10/364,886, filed on Feb. 11, 2003, now abandoned.
(60) Provisional application No. 60/356,672, filed on Feb. 11, 2002.
(60) Provisional application No. 60/356,657, filed on Feb. 11, 2002.

(51) Int. Cl. A61F 9/008

(52) U.S. Cl. 606/5

(57) ABSTRACT

The present invention comprises a closed loop system and method for assessing a performance of a refractive surgical system that is capable of correcting lower and higher order aberrations of the eye. In one embodiment, the refractor surgical system comprises a corneal re-shaping laser system and a refractor system that is capable of measuring low and higher order aberrations of the eye. A software application is capable of transforming the measurements of the refractor system to a treatment plan to control and guide the corneal re-shaping laser system. The systems and methods of the present invention may include a lens that is created by the corneal reshaping laser system and can be measured by the refractor system.

### CROSS-REFERENCES TO RELATED APPLICATIONS

The present application is a continuation of U.S. patent application Ser. No. 10/364,886 filed Feb. 11, 2003, which application claims benefit of U.S. Provisional Patent Appln. No. 60/356,672 filed Feb. 11, 2002; the full disclosures of which are incorporated herein by reference in their entirety.

The present application is also related to U.S. Provisional Patent Appln No. 60/356,658 entitled “Apparatus and Method for Determining Relative Positional and Rotational Offsets between a First and Second Imaging Device,” and Provisional Patent Appln. No. 60/356,657 entitled “Method and Device for Calibrating an Optical Wavefront System,” both of which were filed on Feb. 11, 2002; the full disclosures of which are incorporated herein by reference in their entirety.

### BACKGROUND OF THE INVENTION

The present invention is generally related to design, manufacture, and measurement of lenses with aberrations. The invention provides devices, systems, and methods for measurement and correction of optical errors of optical systems, and is particularly well-suited for validating refractive optical corrections of the eye.

Known laser eye surgery procedures generally employ an ultraviolet or infrared laser to remove a microscopic layer of stromal tissue from the cornea of the eye. The laser typically removes a selected shape of the corneal tissue, often to correct refractive errors of the eye. Ultraviolet laser ablation results in photodecomposition of the corneal tissue, but generally does not cause significant thermal damage to adjacent and underlying tissues of the eye. The irradiated molecules are broken into smaller volatile fragments photochemically, directly breaking the intermolecular bonds.

Laser ablation procedures can remove the targeted stroma of the cornea to change the cornea's contour for varying purposes, such as for correcting myopia, hyperopia, astigmatism, and the like. Control over the distribution of ablation energy across the cornea may be provided by a variety of systems and methods, including the use of ablatable masks, fixed and moveable apertures, controlled scanning systems, eye movement tracking mechanisms, and the like. In known systems, the laser beam often comprises a series of discrete pulses of laser light energy, with the total shape and amount of tissue removed being determined by the shape, size, location, and/or number of laser energy pulses impinging on the cornea. A variety of algorithms may be used to calculate the pattern of laser pulses used to reshape the cornea so as to correct a refractive error of the eye. Known systems make use of a variety of forms of lasers and/or laser energy to effect the correction, including infrared lasers, ultraviolet lasers, femtosecond lasers, wavelength multiplied solid-state lasers, and the like. Alternative vision correction techniques make use of radial incisions in the cornea, intraocular lenses, removable corneal support structures, and the like.

Known corneal correction treatment methods have generally been successful in correcting standard vision errors, such as myopia, hyperopia, astigmatism, and the like. However, as with all successes, still further improvements would be desirable. Toward that end, wavefront measurement systems are now available to measure the refractive characteristics of a particular patient's eye. By customizing an ablation pattern based on wavefront measurements, it may be possible to correct minor aberrations so as to reliably and repeatedly provide visual acuity greater than 20/20.

Known methods for calculation of a customized ablation pattern using wavefront sensor data generally involve mathematically modeling an optical property of the eye using series expansion techniques. More specifically, Zernike polynomials have been employed to model the wavefront surface error map of the eye. Coefficients of the Zernike polynomials are derived through known fitting techniques, and the optical correction procedure is then determined using the shape of the wavefront indicated by the mathematical series expansion model.

In order to properly use these laser ablation algorithms, the laser beam delivery system typically should be calibrated. Calibration of the laser system helps ensure removal of the intended shape and quantity of the corneal tissue so as to provide the desired shape and refractive power modification to the patient's cornea. For example, deviation from a desired laser beam shape or size, such as the laser beam exhibiting a non-symmetrical shape or an increased or decreased laser beam diameter, may result in tissue ablation at an undesired location on the patient's cornea which in turn leads to less than ideal corneal sculpting results. As such, it is beneficial to know the shape and size profiles of the laser beam so as to accurately sculpt the patient's cornea through laser ablation. In addition, it is usually desirable to test for acceptable levels of system performance. For example, such tests can help ensure that laser energy measurements are accurate. Ablations of plastic test materials are often performed prior to laser surgery to calibrate the laser energy and ablation shape of the laser beam delivery system. Although such laser ablation calibration techniques are fairly effective, in some instances, alternative methods for laser energy and beam shape calibration may be advantageous.

Work in connection with the present invention suggests that the known methodology for evaluation of a laser ablation treatment protocol based on wavefront sensor data may be less than ideal. The known laser calibration and test methods may result in errors or “noise” which can lead to a less than ideal optical correction. Furthermore, the known calibration techniques are somewhat indirect, and may lead to unnecessary errors in ablation, as well as a lack of understanding of the physical correction performed.

In light of the above, it would be desirable to provide improved optical correction techniques, particularly for use in procedures for correcting aberrant refractive properties of an eye.

### SUMMARY OF THE INVENTION

The present invention comprises a system and method for testing a performance of a laser system with a closed loop system.

In accord with one aspect, the present invention provides a close looped method of testing a performance of a laser system. The method comprises ablating a surface of a material (e.g., lens material) with a predetermined optical surface. The ablated optical surface is measured and the measured ablated optical surface is compared to the predetermined optical surface.

The predetermined optical surface and the ablated optical surface may be mathematically represented by Zernike polynomial series. The Zernike polynomial series may be compared to determine the differences between the predetermined optical surface and the ablated surface. As can be appreciated, in other alternative embodiments, the optical surfaces may be represented by Taylor or other polynomial series, a surface elevation map, gradient fields, or the like.

In another aspect, the present invention provides a closed looped system for testing a performance of a laser system. The system comprises a laser system that ablates a predetermined optical surface. A wavefront measurement system measures the ablated optical surface, and a processor compares the measured optical surface to the predetermined optical surface.

The predetermined optical surface may be represented by a wavefront elevation surface and may be mathematically defined by a Zernike polynomial series. The processor may be configured to measure the wavefront elevation surface of the ablated optical surface and calculated a corresponding Zernike polynomial series. The Zernike polynomial series of the predetermined optical surface and the measured ablated optical surface may be compared to measure the performance of the system.

In another embodiment, the present invention provides a system for testing a performance of a laser system. The system comprises means for ablating a predetermined optical surface in a surface of a lens material. The ablated optical surface is analyzed with measuring means to determine a measured optical surface of the lens material. The measured optical surface and the predetermined optical surface are compared with comparing means to test the performance of the laser system.

These and other advantages of the invention will become more apparent from the following detailed description of the invention when taken in conjunction with the accompanying exemplary drawings.

### BRIEF DESCRIPTION OF THE DRAWINGS

FIG. 1 is a perspective view of a laser ablation system for incorporating the invention;

FIG. 2 schematically illustrates a system for measuring a wavefront elevation surface in an aspect of an embodiment of the current invention;

FIG. 2A schematically illustrates an alternate wavefront sensor system suitable for use with the method of the present invention;

FIG. 3 schematically illustrates a test fixture for measuring ablated surfaces in accordance with an aspect of an embodiment of the current invention;

FIG. 3A schematically illustrates an ablated optical surface on a plastic lens that has markings for orientation;

FIG. 4 schematically illustrates a Hartman Shack sensor pattern for a measured ablation surface in accordance with an aspect of an embodiment of the present invention;

FIG. 5 lists non-normalized Zernike polynomial basis functions through 6^{th} radial order in both polar and Cartesian form with standard double notation;

FIG. 6 schematically illustrates an embodiment of a closed loop method and system for comparing a theoretical aberration to a measured aberration from an ablation shape correcting the theoretical aberration;

FIG. 7 schematically illustrates a comparison of the Zernike coefficients of a theoretical wavefront elevation surface to the coefficients of another wavefront elevation surface from a measured ablation intended to correct the aberrations of the theoretical surface, in accord with an embodiment of the invention;

FIG. 8 schematically illustrates a comparison of the Zernike coefficients of another theoretical wavefront elevation surface to the Zernike coefficients of another wavefront elevation surface from a measured ablation intended to correct the aberrations of the theoretical surface, in accord with an embodiment of the invention;

FIG. 9 graphically illustrates a comparison of a theoretical wavefront elevation surface map and a measured wavefront elevation surface map intended to correct the aberrations of the theoretical wavefront elevation surface, in accord with an embodiment of the invention;

FIG. 10 graphically illustrates a comparison of another theoretical wavefront elevation surface map and another measured wavefront elevation surface map intended to correct the aberrations of the theoretical wavefront elevation surface, in accord with an embodiment of the invention;

FIG. 11 illustrates a simulation of translational and rotational displacement of a measured wavefront elevation surface intended to correct a theoretical wavefront elevation surface by listing the Zernike coefficients of the theoretical surface, the coefficients of the surface that is displaced and rotated in the simulation, and the coefficients actually measured from a corrective ablation in accord with an embodiment of the invention;

FIG. 12 illustrates a synthetic spot pattern that is used to test the closed loop system in accordance with an embodiment of the invention; and

FIG. 13 illustrates a flow chart in accord with an embodiment of the invention that is used to determine a patient treatment in response to a closed loop comparison of a theoretical wavefront elevation surface and a measured wavefront elevation surface.

### DETAILED DESCRIPTION OF THE INVENTION

The present invention is particularly useful for enhancing the accuracy and efficacy of laser eye surgical procedures, such as photorefractive keratectomy (PRK), phototherapeutic keratectomy (PTK), laser in situ keratomileusis (LASIK), and the like. Preferably, the present invention can provide enhanced optical accuracy of refractive procedures by improving the methodology for calibrating, testing and validating a corneal ablation or other refractive treatment program. Hence, while the system and methods of the present invention are described primarily in the context of a laser eye surgery system, it should be understood the techniques of the present invention may be adapted for use in alternative eye treatment procedures and systems such as spectacle lenses, intraocular lenses, contact lenses, corneal ring implants, collagenous corneal tissue thermal remodeling, and the like.

The techniques of the present invention can be readily adapted for use with existing laser systems, wavefront sensors, and other optical measurement devices. By providing a more direct (and hence, less prone to noise and other error) methodology for measuring and correcting errors of an optical system, the present invention may facilitate sculpting of the cornea so that treated eyes regularly exceed the normal 20/20 threshold of desired vision.

Wavefront sensors will typically measure aberrations and other optical characteristics of an entire optical tissue system. The data from such a wavefront sensor may be used to generate an optical surface from an array of optical gradients. The measured array of optical gradients comprises a gradient field of a measured optical surface, and the measured gradient field is used to reconstruct a wavefront elevation surface map. It should be understood that the optical surface need not precisely match an actual tissue surface, as the gradients will show the effects of aberrations which are actually located throughout the ocular tissue system. Nonetheless, corrections imposed on an optical tissue surface so as to correct the aberrations derived from the gradients should correct the optical tissue system. As used herein terms such as “an optical tissue surface” may encompass a theoretical tissue surface (derived, for example, from wavefront sensor data), an actual tissue surface, and/or a tissue surface formed for purposes of treatment (for example, by incising corneal tissues so as to allow a flap of the corneal epithelium and stroma to be displaced and expose the underlying stroma during a LASIK procedure).

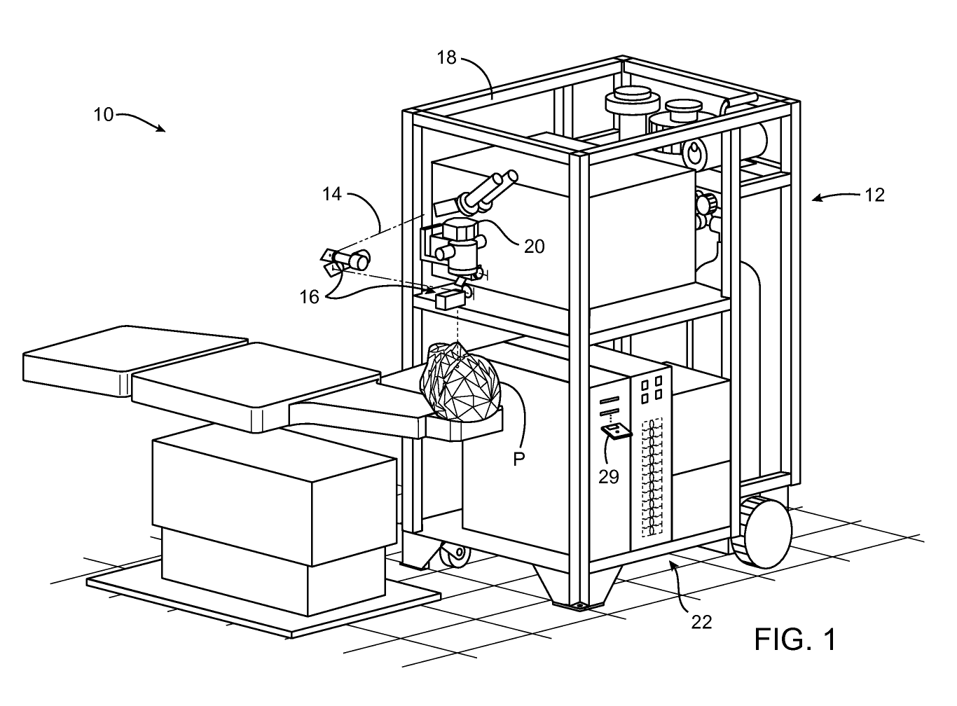

Referring now to FIG. 1, a laser eye surgery system 10 of the present invention includes a laser 12 that produces a laser beam 14. Laser 12 is optically coupled to laser delivery optics 16, which directs laser beam 14 to an eye of patient P. A delivery optics support structure (not shown here for clarity) extends from a frame 18 supporting laser 12. A microscope 20 is mounted on the delivery optics support structure, the microscope often being used to image a cornea of the eye.

Laser 12 generally comprises an excimer laser, ideally comprising an argon-fluorine laser producing pulses of laser light having a wavelength of approximately 193 nm. Laser 12 will preferably be designed to provide a feedback stabilized fluence at the patient's eye, delivered via delivery optics 16. The present invention may also be useful with alternative sources of ultraviolet or infrared radiation, particularly those adapted to controllably ablate the corneal tissue without causing significant damage to adjacent and/or underlying tissues of the eye. In alternate embodiments, the laser beam source employs a solid state laser source having a wavelength between 193 and 215 nm as described in U.S. Pat. Nos. 5,520,679, and 5,144,630 to Lin and 5,742,626 to Mead, the full disclosures of which are incorporated herein by reference. In another embodiment, the laser source is an infrared laser as described in U.S. Pat. Nos. 5,782,822 and 6,090,102 to Telfair, the full disclosure of which is incorporated herein by reference. Hence, although an excimer laser is the illustrative source of an ablating beam, other lasers may be used in the present invention.

Laser 12 and delivery optics 16 will generally direct laser beam 14 to the eye of patient P under the direction of a computer 22. Computer 22 will often selectively adjust laser beam 14 to expose portions of the cornea to the pulses of laser energy so as to effect a predetermined sculpting of the cornea and alter the refractive characteristics of the eye. In many embodiments, both laser 14 and the laser delivery optical system 16 will be under control of processor 22 to effect the desired laser sculpting process, with the processor effecting (and optionally modifying) the pattern of laser pulses. The pattern of pulses may by summarized in machine readable data of tangible media 29 in the form of a treatment table, and the treatment table may be adjusted according to feedback input into processor 22 from an automated image analysis system (or manually input into the processor by a system operator) in response to feedback data provided from an ablation monitoring system feedback system. Such feedback might be provided by integrating the wavefront measurement system described below with the laser treatment system 10, and processor 22 may continue and/or terminate a sculpting treatment in response to the feedback, and may optionally also modify the planned sculpting based at least in part on the feedback.

A laser treatment table includes the horizontal and vertical position of the laser beam on the eye for each laser beam pulse in a series of pulses. Preferably, the diameter of the beam varies during the treatment from about 0.65 mm to 6.5 mm. The treatment table typically includes several hundred pulses and the number of laser beam pulses varies with the amount of material removed and laser beam diameters employed by the laser treatment table. The computer program that generates a laser treatment table selects a pattern of laser beam pulses that will create an optical surface shape in plastic that makes the desired wavefront elevation surface as light passes through the material.

For systems measuring the closed loop system properties in plastic, a flat plastic lens is preferred. Although flat plastic is preferred, other plastic shapes may be ablated including curved plastic having a surface radius of curvature of about 7.5 mm. The laser treatment table is calculated using the shape of material removed with each pulse of the laser beam, and the shape of material removed with an individual pulse of a laser beam is referred to as a crater. The shape of material removed at each beam diameter is also referred to as basis data. For a rotationally symmetric laser beam the basis data are rotationally averaged. The optical surface shape resulting from material removal during a laser treatment is calculated by adding the craters of material removed by each pulse of the laser beam in the treatment table. Preferably, the calculated optical surface shape resulting from material removal matches the intended optical surface shape to within a desirable tolerance averaging about a quarter of a wavelength of visible light, or about 0.2 μm over the ablated surface. A calculation of a treatment table is more fully described in U.S. patent application Ser. No. 09/805,737 filed on Mar. 13, 2001 (now U.S. Pat. No. 6,673,062), and published on Sep. 20, 2001 under the PCT as Publication No. WO01/67978; the full disclosures of which are incorporated herein by reference.

The relationship between the depth of material removed and a corresponding change in the optical surface is related to the index of refraction of the material removed. For example, the depth of material to be removed can be calculated by dividing the corrective wavefront elevation surface map by the quantity (n−1) where n is the index of refraction of the material. This relation is an application of Fermat's principal of least time, known for over 300 years. The index of refraction of the cornea is 1.377 and the index of refraction of plastic is about 1.5. An embodiment of the present invention uses VISX calibration plastic having an index or refraction of 1.569. This material is available from VISX, Inc. Santa Clara, Calif. An embodiment of a technique for such a calculation of ablation depth is also described in U.S. Pat. No. 6,271,914, the full disclosure of which is herein incorporated by reference.

Laser beam 14 may be adjusted to produce the desired sculpting using a variety of alternative mechanisms. The laser beam 14 may be selectively limited using one or more variable apertures. An exemplary variable aperture system having a variable iris and a variable width slit is described in U.S. Pat. No. 5,713,892, the full disclosure of which is incorporated herein by reference. The laser beam may also be tailored by varying the size and offset of the laser spot from an axis of the eye, as described in U.S. Pat. No. 5,683,379, and as also described in U.S. patent application Ser. No. 08/968,380 filed Nov. 12, 1997 (now issued as U.S. Pat. No. 6,203,539); and 09/274,999 filed Mar. 22, 1999 (now issued as U.S. Pat. No. 6,347,549); the full disclosures of which are incorporated herein by reference.

Still further alternatives are possible, including scanning of the laser beam over a surface of the eye and controlling the number of pulses and/or dwell time at each location, as described, for example, by U.S. Pat. No. 4,665,913 (the full disclosure of which is incorporated herein by reference); using masks in the optical path of laser beam 14 which ablate to vary the profile of the beam incident on the cornea, as described in U.S. patent application Ser. No. 08/468,898 filed Jun. 6, 1995 (the full disclosure of which is incorporated herein by reference); hybrid profile-scanning systems in which a variable size beam (typically controlled by a variable width slit and/or variable diameter iris diaphragm) is scanned across the cornea; or the like. The computer programs and control methodology for these laser pattern tailoring techniques are well described in the patent literature.

Additional components and subsystems may be included with laser system 10, as should be understood by those of skill in the art. For example, spatial and/or temporal integrators may be included to control the distribution of energy within the laser beam, as described in U.S. Pat. No. 5,646,791, the disclosure of which is incorporated herein by reference. An ablation effluent evacuator/filter, and other ancillary components of the laser surgery system which are not necessary to an understanding of the invention, need not be described in detail for an understanding of the present invention.

Processor 22 may comprise (or interface with) a conventional PC system including the standard user interface devices such as a keyboard, a display monitor, and the like. Processor 22 will typically include an input device such as a magnetic or optical disk drive, an internet connection, or the like. Such input devices will often be used to download a computer executable code from a tangible storage media 29 embodying any of the methods of the present invention. Tangible storage media 29 may take the form of a floppy disk, an optical disk, a data tape, a volatile or non-volatile memory, or the like, and the processor 22 will include the memory boards and other standard components of modern computer systems for storing and executing this code. Tangible storage media 29 may optionally embody wavefront sensor data, wavefront gradients, a wavefront elevation map, a treatment map, and/or an ablation table.

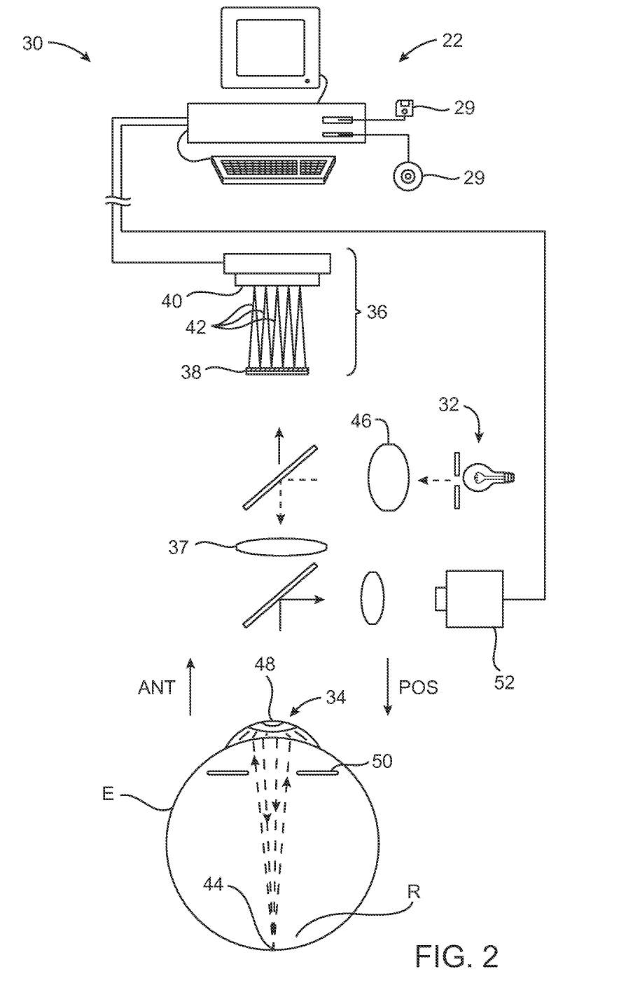

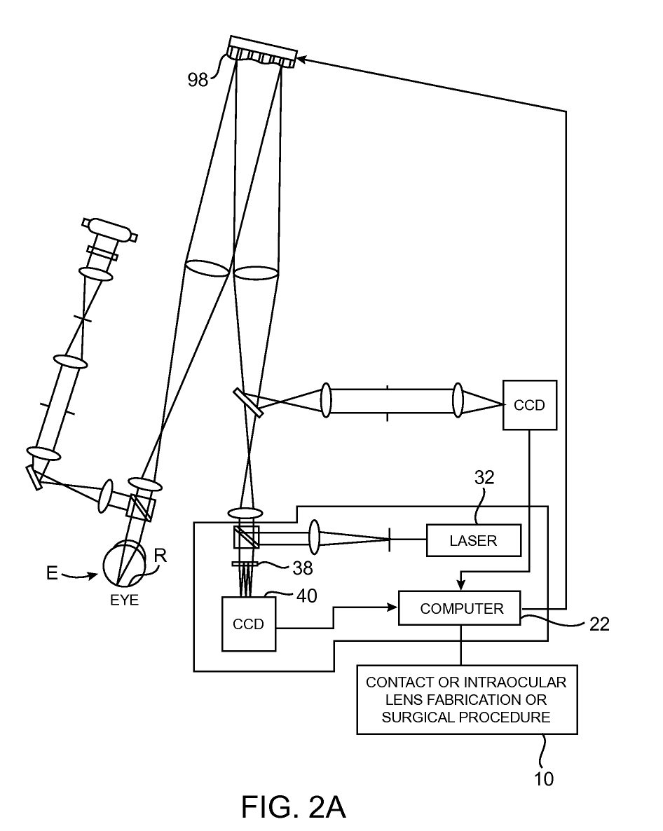

Referring now to FIG. 2, an exemplary wavefront sensor system 30 is schematically illustrated in simplified form. In very general terms, wavefront system 30 includes an image source 32 which projects a source image through optical tissues 34 of eye E and so as to form an image 44 upon a surface of retina R. The image from retina R is transmitted by the optical system of the eye (e.g., optical tissues 34) and imaged onto a wavefront sensor 36 by system optics 37. The wavefront sensor 36 communicates signals to computer 22 for determination of a corneal ablation treatment program. Computer 22 may be the same computer which is used to direct operation of the laser surgery system 10, or at least some or all of the computer components of the wavefront sensor system and laser surgery system may be separate. Data from wavefront sensor 36 may be transmitted to a separate laser system computer via tangible media 29, via an I/O port, via an networking connection such as an intranet or the Internet, or the like.

Wavefront sensor 36 generally comprises a lenslet array 38 and an image sensor 40. As the image from retina R is transmitted through optical tissues 34 and imaged onto a surface of image sensor 40 and an image of the eye pupil P is similarly imaged onto a surface of lenslet array 38, the lenslet array separates the transmitted image into an array of beamlets 42, and (in combination with other optical components of the system) images the separated beamlets on the surface of sensor 40. Sensor 40 typically comprises a charged couple device or “CCD,” and senses the characteristics of these individual beamlets, which can be used to determine the characteristics of an associated region of optical tissues 34. In particular, where image 44 comprises a point or small spot of light, a location of the transmitted spot as imaged by a beamlet can directly indicate a local gradient of the associated region of optical tissue.

Eye E generally defines an anterior orientation ANT and a posterior orientation POS. Image source 32 generally projects an image in a posterior orientation through optical tissues 34 onto retina R as indicated in FIG. 2. Optical tissues 34 again transmit image 44 from the retina anteriorly toward wavefront sensor 36. Image 44 actually formed on retina R may be distorted by any imperfections in the eye's optical system when the image source is originally transmitted by optical tissues 34. Optionally, image source projection optics 46 may be configured or adapted to decrease any distortion of image 44.

In some embodiments, image source optics may decrease lower order optical errors by compensating for spherical and/or cylindrical errors of optical tissues 34. Higher order optical errors of the optical tissues may also be compensated through the use of an adaptive optic element, such as a deformable mirror. Use of an image source 32 selected to define a point or small spot at image 44 upon retina R may facilitate the analysis of the data provided by wavefront sensor 36. Distortion of image 44 may be limited by transmitting a source image through a central region 48 of optical tissues 34 which is smaller than a pupil 50, as the central portion of the pupil may be less prone to optical errors than the peripheral portion. Regardless of the particular image source structure, it will be generally be beneficial to have well-defined and accurately formed image 44 on retina R.

While the method of the present invention will generally be described with reference to sensing of an image 44, it should be understood that a series of wavefront sensor data readings may be taken. For example, a time series of wavefront data readings may help to provide a more accurate overall determination of the ocular tissue aberrations. As the ocular tissues can vary in shape over a brief period of time, a plurality of temporally separated wavefront sensor measurements can avoid relying on a single snapshot of the optical characteristics as the basis for a refractive correcting procedure. Still further alternatives are also available, including taking wavefront sensor data of the eye with the eye in differing configurations, positions, and/or orientations. For example, a patient will often help maintain alignment of the eye with wavefront sensor system 30 by focusing on a fixation target, as described in U.S. Pat. No. 6,004,313, the full disclosure of which is incorporated herein by reference. By varying a focal position of the fixation target as described in that reference, optical characteristics of the eye may be determined while the eye accommodates or adapts to image a field of view at a varying distance.

The location of the optical axis of the eye may be verified by reference to the data provided from a pupil camera 52. In the exemplary embodiment, a pupil camera 52 images pupil 50 so as to determine a position of the pupil for registration of the wavefront sensor data relative to the optical tissues.

An alternative embodiment of a wavefront sensor system is illustrated in FIG. 2A. The major components of the system of FIG. 2A are similar to those of FIG. 2. Additionally, FIG. 2A includes an adaptive optical element 98 in the form of a deformable mirror. The source image is reflected from deformable mirror 98 during transmission to retina R, and the deformable mirror is also along the optical path used to form the transmitted image between retina R and imaging sensor 40. Deformable mirror 98 can be controllably deformed to limit distortion of the image formed on the retina or of subsequent images formed of the images formed on the retina, and may enhance the accuracy of the wavefront data. The structure and use of the system of FIG. 2A are more fully described in U.S. Pat. No. 6,095,651, the full disclosure of which his incorporated herein by reference.

The components of an embodiment of a wavefront system for measuring the eye and ablations comprise elements of a VISX WaveScan™, available from VISX, INCORPORATED of Santa Clara, Calif. One embodiment includes a WaveScan with a deformable mirror as described above. An alternate embodiment of a wavefront measuring device is described in U.S. Pat. No. 6,271,915, the full disclosure of which is incorporated herein by reference.

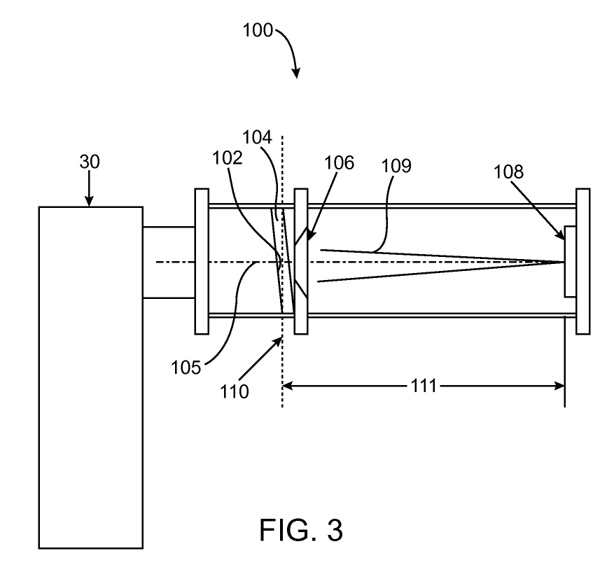

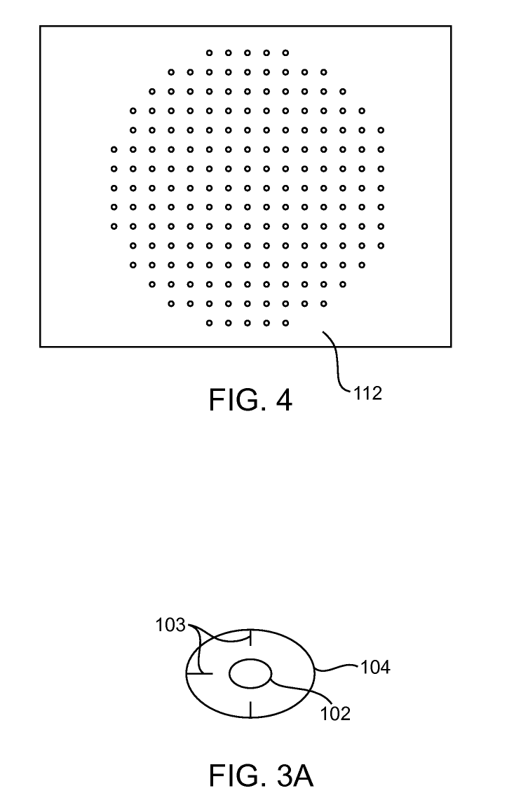

A test fixture 100 for measuring the aberrations of the ablated optical surface 102 formed in a plate of an optically transparent plastic material 104 is shown in FIGS. 3 and 3A. An optical measurement system 30 as described above is configured to measure gradients of a wavefront field formed by light passing through an ablated optical surface 102. The ablated optical surface 102 having aberrations is placed on the test fixture 100 that includes a pupil 106 and a reflecting surface 108. The distance between the pupil and reflecting surface is accurately controlled and is preferably about 166 mm, although other suitable distances may be used. The ablated optical surface 102 is placed adjacent to the pupil 106. The optically transparent plate may be mounted with a slight tilt relative to an optical axis 105 of system 30, as is illustrated in FIG. 3. A small tilt of the measured optical surface 102 relative to the system optical axis 105 deflects reflections of the incoming measurement beam from the measured optical surface 102 and back surface of transparent plate 104. The ablation may be centered in pupil 106 of the fixture 100. The test fixture is mounted on the wavefront measurement system aligned with the system optical axis 105.

Preferably the same wavefront sensor or a substantially similar wavefront sensor is used to measure the ablated plastic and measure the eye. Alternatively, another type of wavefront sensor that is fundamentally similar to the wavefront sensor used to measure the eye may be employed to measure the ablated optical surface. As used herein substantially similar wavefront sensors encompass wavefront sensors having similar operating principals and functional components such as a lenslet array, a focused light beam and the like. As used herein fundamentally similar wavefront systems encompass wavefront systems employing a similar fundamental operating principal, for example measuring a gradient field made by passing light through an optical surface. Another example of a similar fundamental operating principal is measuring an optical surface with a light beam interference pattern by interferometry. Examples of wavefront sensors measuring gradient fields of light passing through the eye include, for example, systems using the principles of ray tracing aberrometry, Tscherning aberrometry, and dynamic skiascopy. The above systems are available from TRACEY TECHNOLOGIES of Bellaire, Tex.; WAVELIGHT of Erlangen, Germany; and NIDEK, INC. of Fremont, Calif., respectively. Other examples of a systems measuring a gradient field of an eye include spatially resolved refractometers as described in U.S. Pat. Nos. 6,099,125; 6,000,800; and 5,258,791, the full disclosures of which are incorporated herein by reference.

An alternate embodiment of the closed loop system uses a first device to measure the eye and a second device to measure the ablated optical surface, wherein the first device and the second device employ different fundamental operating principals. For example, the eye is measured by a device that measures a gradient field of light passing through the eye, and the ablated optical surface is measured by an interferometer. Alternatively, the ablated optical surface may be measured by a diamond stylus profilometer or a moiré fringe projection system, or other surface profile technology.

The wavefront sensor 30 may include internal lenses that compensate for much of the refractive error of the eye. If such lenses are not present, then a focusing lens (not shown) may be added to the test fixture 100 between measurement system 30 and reflecting surface 108. These lenses are adjusted to form a focused beam of light 109 on reflecting surface 108. The focused beam of light 109 is reflected back from the surface 108 and passes through the pupil 106 and the optical surface 102 formed in a plate of an optically transparent plastic material 104 that may optionally include orientation markings 103. The wavefront system includes a measurement plane 110 where an eye is positioned for measurement. The optical surface 102 is positioned at measurement plane 110 near the pupil 106. A distance 111 between ablated optical surface 102 and reflecting surface 108 is measured. The distance 111 is related to the inverse of the spherical defocus refractive error of test fixture 100. For a distance 111 of ⅙ of a meter the spherical defocus refractive error is +6 Diopters. A measurement is taken through the ablated optical surface 102 with wavefront sensor 30. The wavefront sensor 30 forms an array of spots 112 of light energy on an electronic sensor as illustrated in FIG. 4.

The positions of the spots are related to the gradient field of the wavefront elevation surface of a light beam passed through ablated surface 102, and the positions of the spots are used to calculate the gradient of the wavefront corresponding to each spot. The gradient values from each spot are used to reconstruct the wavefront elevation surface map of the ablated optical surface 102.

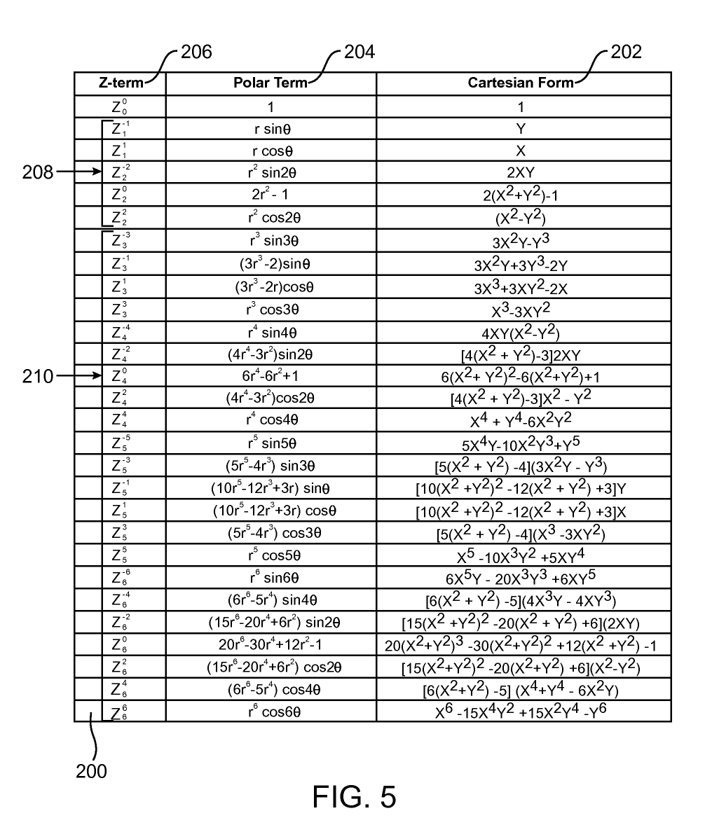

The wavefront of ablated optical surface 102 is preferably represented as a Zernike polynomial series 200 as illustrated in FIG. 5. The Zernike polynomials are illustrated in Cartesian 202 and polar 204 form for each Z term 206. The terms are described with a standard double notation. The double notation describes the radial and angular order of each term. The superscript of the double notation describes the angular order, and the subscript of the double notation describes the radial order. Terms having a radial order of 1 and 2 correspond to aberrations that are corrected with eyeglass prescriptions and encompass low order or lower order aberrations 208. Radial terms above second order encompass high order or higher order aberrations 210. Although the radial and angular Zernike terms described in FIG. 5 are described to 6^{th} order, this description is by way of example, and these Zernike terms can be described and fitted to a measured gradient field from a measured ablated optical surface 102 to any arbitrarily chosen order or precision (e.g., tenth order and above).

In alternate embodiments, the wavefront may be represented as a Taylor or other polynomial series. Alternatively, the wavefront elevation surface may be represented as a surface elevation map and may also be represented by the measured gradient field.

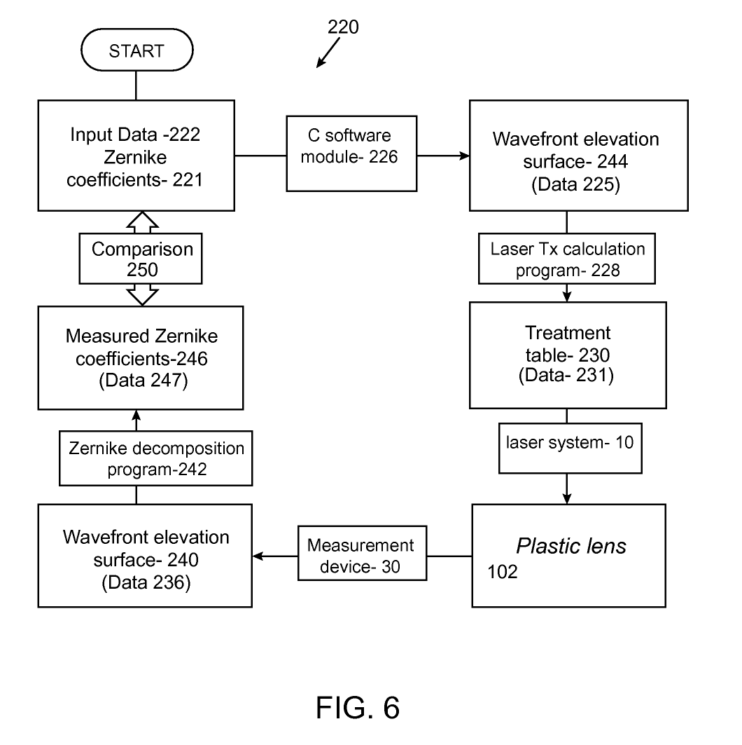

A closed loop system 220 for comparing input data 222 corresponding to an optical aberration and measured ablation data 236 corresponding to an ablated optical surface 102 that corrects the optical aberration in an embodiment of the invention is illustrated in FIG. 6. A set of Zernike coefficients 221 representing a theoretical optical surface are input data 222 to the system 220. Input data 222 to closed loop system 220 includes any suitable data representation of an optical surface including a wavefront measurement of an eye, a set of polynomial coefficients from a wavefront measurement of an eye and a set of gradients from a wavefront measurement. The Zernike coefficients 221 are preferably in the form of a linear combination of basis functions on a unit circle. The coordinate system is preferably right handed with the positive X axis directed to the right along local horizontal and the Z axis directed outward from the eye and so conforms to the standard ophthalmic coordinate system (ISO 8429:1986). The wavefront is preferably defined for a 6 mm optical zone, and sampled on a rectangular grid. The rectangular grid preferably has a spacing of 0.1 mm in horizontal and vertical directions. The Zernike coefficients are converted to data 225 representing an optical wavefront elevation surface 224 having an elevation over the points of the grid. The calculation of the wavefront elevation surface 224 from the Zernike coefficients 221 may be performed with a C software module 226. The diameter of the wavefront elevation surface is typically about 6 mm. To calculate an elevation at a point in the grid, the coordinates of the point and corresponding Zernike coefficients are input into an analytical expression for a linear combination of Zernike polynomials. In this embodiment, the Zernike coefficients are associated with the non normalized Zernike functions. These coefficients may be scaled to give the wavefront surface elevation in microns. This scaling is done for the diameter of the pupil, which is 6 mm in this illustrative embodiment and may be other sizes.

After determining the wavefront elevation surface a laser treatment calculation program 228 analyzes data 231 to calculate a treatment table 230 of laser pulse instructions as described above. The laser treatment table is designed to make an ablated optical surface 102 that corrects for the aberrations described by the wavefront elevation surface 224.

The treatment table is loaded from a tangible media 29 onto laser system 10 by processor 22. In an embodiment, the laser system comprises elements of the VISX Star S3 Excimer Laser System, and the plate 104 comprises calibration plastic available from VISX, Inc., Santa Clara, Calif. A plate of an optically transparent material 104 is ablated with laser system 10 to form an optical ablation surface 102 in the form of a plastic lens.

The ablated optical surface 102 is placed in the calibration fixture 100 as described above. The ablated optical surface 102 is measured with a wavefront measurement device 30 as described above. The wavefront measuring device is preferably a VISX WaveScan, available from VISX, Inc., Santa Clara, Calif. Alternate embodiments may employ other suitable measurement systems as described above. The wavefront measuring device measures the gradient field of the optical surface of a light beam passing through the ablation as described above. The wavefront elevation surface 240 is mathematically constructed from the gradient field as described above. Alternatively, Zernike polynomial coefficients are calculated by integrating the gradient field.

The measured wavefront elevation surface 240 is decomposed with a Zernike decomposition program 242 that calculates data 247 as a series of measured Zernike coefficients 246. In one embodiment, a Matlab program calculates the decomposition with a Gram-Schmidt orthogonalization method. Matlab™ is available from THE MATHWORKS, INC. of Natick, Mass. In an alternate embodiment, another suitable computer program such as a C computer program may be written to perform the decomposition. In further embodiments the Zernike coefficients are calculated directly from the measured gradient field as described above.

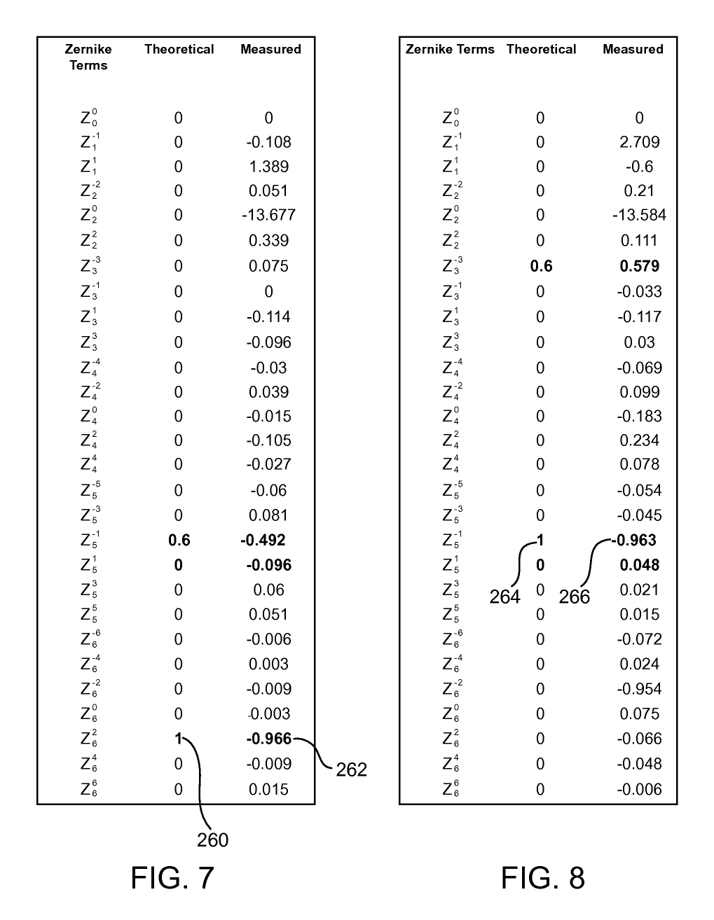

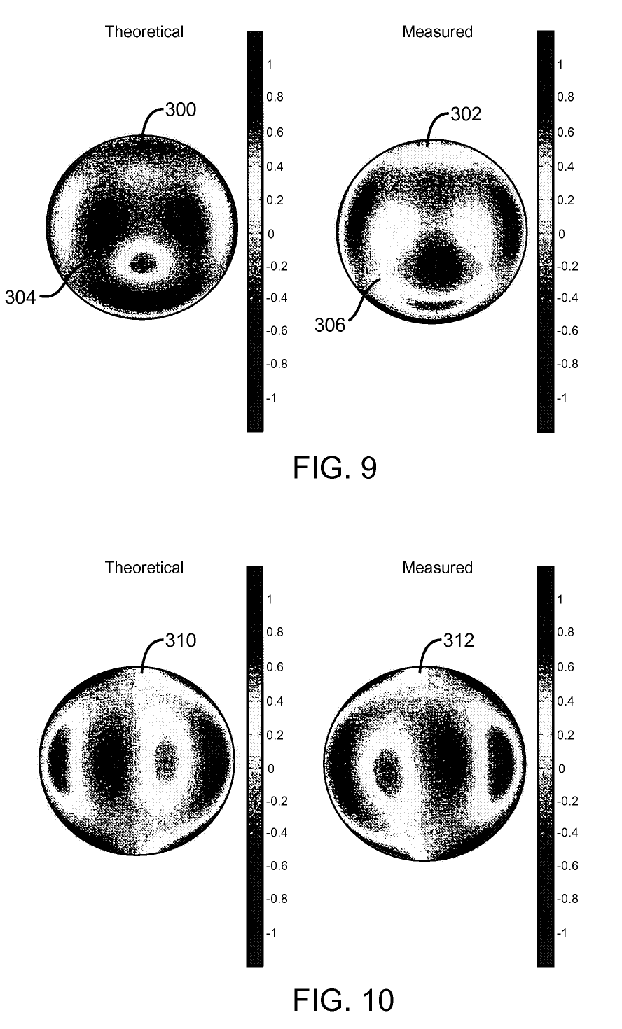

A comparison 250 of the input Zernike coefficients with the measured Zernike coefficients indicates the overall accuracy of the system. The comparison preferably includes a comparison of individual measured Zernike coefficients 262, 266 with a corresponding intended theoretical values 260, 264 of the Zernike coefficient as illustrated in FIGS. 7 and 8 respectively. Other comparisons in addition to polynomial coefficient comparisons include comparisons of graphic illustrations of theoretical wavefront elevation surfaces 300, 310 and measured wavefront elevation surfaces 302, 312 that are compared by a user of system 10 as illustrated in FIGS. 9 and 10 respectively.

By way of illustrative example two wavefront elevation surfaces that are tested with the closed loop system are a first surface S1 and a second surface S2. Equations that describe surfaces elevations of S1 and S2 (in microns) are:

S1=0.6*Z₅⁻¹+1.0*Z₆²

S2=0.6*Z₃⁻³+1.0*Z₅⁻¹

The above equations for S1 and S2 are input as a theoretical surface into the closed loop system 220. For surfaces S1 and S2, the resulting measured coefficients for the ablated optical surface are illustrated in FIGS. 7 and 8 respectively. In FIGS. 7 and 8, individual measured and theoretical Zernike coefficients are listed for each term. In these embodiments, the measured values are expected to have the same magnitude as the input values and be of the opposite sign because the wavefront system measures an error of an eye and the lens is ablated to correct the error of an eye. In other words, the sum of the input wavefront elevation surface and output wavefront elevation surface is zero in a closed loop system with no measurable error. Raw measured data are illustrated in FIGS. 7 and 8. Several low order coefficients are shown to have non zero values. For example term Z₂⁰ has a value of −13.7 and −13.6 in FIGS. 7 and 8 respectively. This value corresponds to an intentional spherical defocus in the wavefront system 30 during the measurement of the optical surface on the test fixture as described above. The illustrative terms corresponding to Z₁⁻¹ and Z₁¹ are non-zero because of the tip and tilt introduced into the system in order to remove the direct beam reflection, as discussed above, and therefore are not be considered in the final comparison. The remaining coefficients represent signals and noise produced by each step of the process.

In FIGS. 9 and 10 theoretical wavefront surface elevation maps 300, 310 are illustrated graphically adjacent to measured corrective wavefront elevation surface maps 302, 312 respectively. The illustration of the measured wavefront elevation surface maps 302, 312 selectively include the high order terms as described above. The appearances of theoretical 300 and measured 302 wavefront elevation surface maps are in the form of figurines 304 and 306 respectively. The figurines are in the form of a happy face, in particular a happy face of an animal, and more particularly in the form of a happy animal of species canisfamiliaris also known as a “Happy Dog.” The coefficients of the Zernike polynomial series are selected to form a Happy Dog figurine when represented as a wavefront elevation surface and ablated in a material.

In other embodiments, the comparison includes an addition of the theoretical wavefront elevation surface to the measured wavefront elevation surface to produce a wavefront elevation error surface map that directly indicates the errors determined from the comparison, and a root mean square value of the error over the error surface map is calculated and reported to an operator of a system.

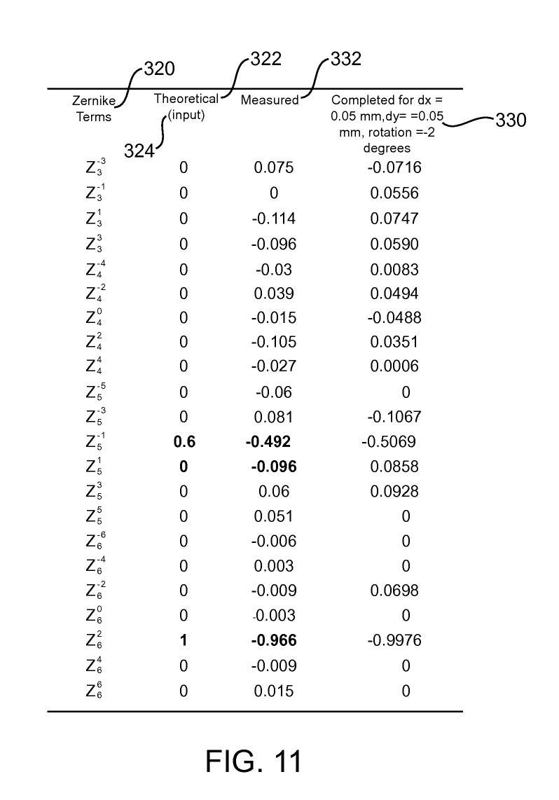

In an embodiment of the invention, degradation to the measured ablated optical surface caused by alignment error is simulated. A result of a simulation is illustrated in FIG. 11. Zernike terms 320 are listed for data 324 representing a theoretical surface 322 input into closed loop system 320. The simulation is achieved by shifting and rotating the theoretical surface 322 and inputting this shifted and rotated surface into the closed loop system 220 as a measured wavefront elevation surface at 240. The output coefficients 330 of the shifted and rotated elevation surface are illustrated adjacent to coefficients 332 illustrating a measured ablated optical surface 102 in FIG. 11.

Rotational misalignment between the placement of the ablated optical surface lens 102 under the laser 10 and the wavefront measurement device 30 causes some of the magnitude of the sine term (Z₅⁻¹) to be transferred to the cosine term (Z₅¹) in surface S1. It is easy to show this effect in the polar form of Zernike functions:

A*f(r)*cos(θ+δ)=A*f(r)*(cos(δ)cos(θ)−sin(δ)*sin(θ))

A*f(r)*sin(θ+δ)=A*f(r)*(cos(δ)sin(θ)+sin(δ)*cos(θ))

where δ is a rotational misalignment, A is a coefficient, r is a radial coordinate, f(r) is a radial function and θ is an angular coordinate.

Another potential source of error between the theoretical and the measured Zernike values is a translational offset between the placement of the lens under the laser and the wavefront measurement device. The effect of such displacement is computed explicitly from the theoretical surfaces as a function of the amount of displacement (dx, dy). Alternatively, the new Zernike coefficients may be directly computed that characterize the displaced surface. This calculation demonstrates that coefficients that are initially zero have non zero values when the measured wavefront is displaced. As an illustrative example, FIG. 11 illustrates changes to the coefficients of surface S1 for a translation of 0.05 mm in the x direction, −0.05 mm in the y direction and a rotation of −2 degrees. The Zernike coefficients are listed for the data input 324 of theoretical surface S1 (322), the measured coefficients, and the coefficients computed for the theoretical input surface S1 (322) after the surface has been translated and rotated. As can be seen, the values for the computed shifted and rotated surface are similar in magnitude to those found by actual measurement. For both the measured surface 322 and the surface rotated and shifted by simulation 330, the amplitudes of the 6^{th} order Zernike coefficients are typically an order of magnitude smaller than the amplitude of the input signal for those coefficients having a value of zero in the theoretical input wavefront. This simulation illustrates a measured ablation optical surface that is well aligned when measured and illustrates an effect on measurements of slight variations in position.

The closed loop system 220 permits an estimate of error caused by other sources in addition to rotational and positional alignment. For example terms Z₆⁻⁴, Z₆⁴ and Z₆⁰ as illustrated in FIG. 11 show values of zero in rotation and translation of input surface S1 (322) after the translations, and yet these terms have non-zero values for the measured ablated optical surface 332. The amplitudes of the errors in these terms are illustrative of the overall noise level of other components of the system in addition to rotational and translational errors of the wavefront system.

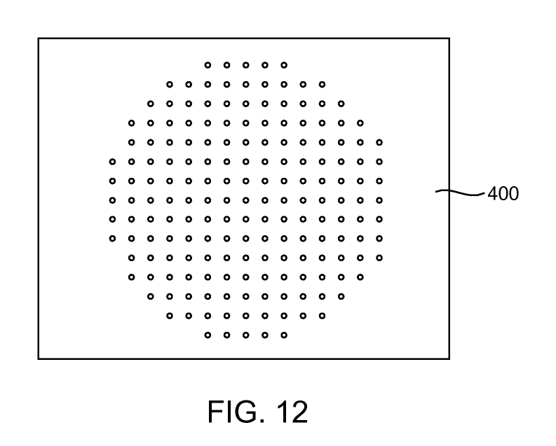

In an embodiment of the invention of FIG. 12, a synthetic image 400 of a Hartmann Shack sensor spot pattern is used with a wavefront measurement system 30. A computer program produces the synthetic spot pattern for a theoretical wavefront surface. For example, the synthetic image 400 illustrates a synthetic spot pattern corresponding to term Z₃⁻³ having a maximum surface elevation amplitude of 1 μm over a 6 mm aperture. Synthetic images similar to image 400 are used to test subsystems of closed loop system 220, for example Zernike decomposition program 242 and software of wavefront measurement system 30.

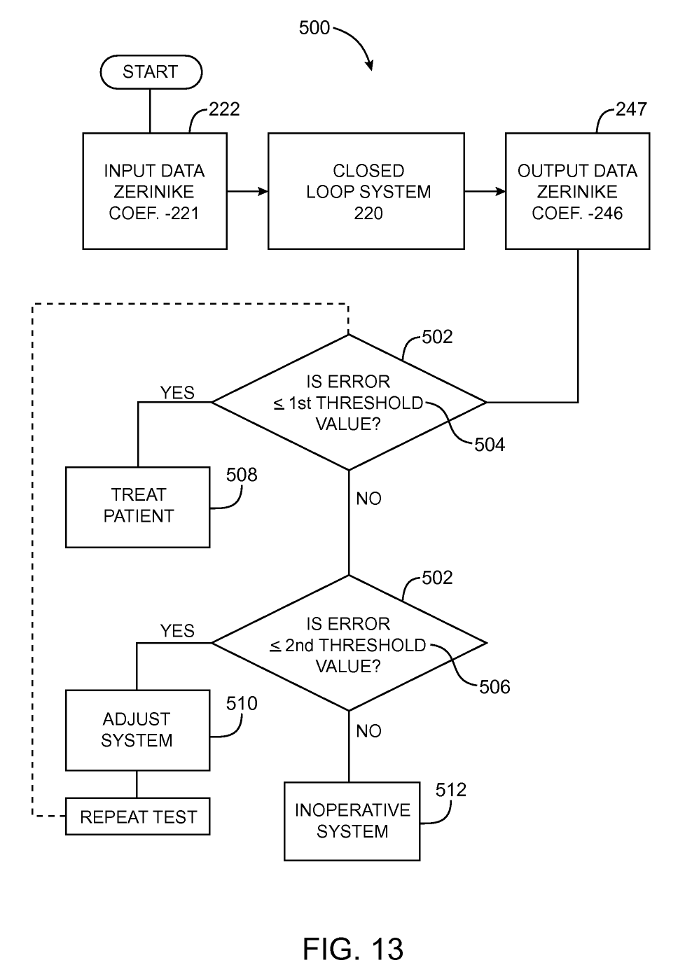

One method of using the system of the present invention is illustrated in FIG. 13. The closed loop system 220 is used prior to laser eye surgery in an embodiment 500 of the invention. A theoretical wavefront surface elevation is represented as Zernike coefficients 221 by data 222 input to the closed loop system 220. The laser system makes an ablated optical surface corrective lens having aberrations, and the ablated optical surface is measured in a wavefront system, as described above. The measured Zernike coefficients 246 of the ablated optical surface are output as data 247 and are compared to the theoretical wavefront surface Zernike coefficients 221 by adding each of the coefficients to produce corresponding error coefficients 502 for each term. If the measured Zernike coefficients 246 are sufficiently close to desired values, the error for each term of the Zernike series is nearly zero and the surgery proceeds 508. If the coefficients of the ablated lens differ from the desired coefficients by more than a first threshold amount 504, but less than a second threshold amount 506, at least one of the components of the system 220 the system is adjusted and another lens ablated. If the coefficients differ by more than a second threshold amount 506 the system is inoperative 512. An adjustment 510 to the system may include adjustments to the laser system including an adjustment to the laser beam energy, an angle and an offset of the ablation pattern, and a magnification scaling of the ablation pattern. Alternatively, the wavefront measurement system may be adjusted, for example by calibration. Once the adjustment is made to the system, the method may be repeated to determine if the measured Zernike coefficients 246 are sufficiently close to desired values.

As described above, the coefficients of an offset ablation are calculated for a given offset and angular orientation of a wavefront surface elevation pattern. By measuring a degradation to a measured ablation pattern as described above, the offset and angular orientation of the ablation pattern are calculated. This offset and orientation are programmed into the laser, and the laser adjusts the ablation pattern. Similarly, if the magnitude of a coefficient of the measured ablation differs from the intended, the laser is programmed to ablate a changed ablation pattern. For example, the changed ablation pattern may be made by adjusting the laser beam energy. Alternatively, the changed ablation pattern may include a change to the basis data used to calculate the treatment table. Similar to rotational and translational alignment errors described above, the closed loop system can detect errors in a scaling of a laser beam offset from a central position. Such an error causes a size of a dimension across the ablated pattern to differ from an expected value. This error appears as a magnification error in a scaling of a size of the ablated shape. The closed loop system detects such errors and adjustments to the scanned laser beam pattern about a central position are made to produce an ablation pattern better matching the intended ablation pattern.

While the specific embodiments have been described in some detail, by way of example and for clarity of understanding, a variety of adaptations, changes, and modifications will be obvious to those of skill in the art. Treatments that may benefit from the invention include intraocular lenses, contact lenses, spectacles and other surgical methods in addition to lasers. Therefore, the scope of the present invention is limited solely by the appended claims.

What is claimed is:

1. A closed loop method for testing a performance of a laser system, the method comprising:
inputting a predetermined optical surface into an ablation system, the predetermined optical surface having high-order optical aberrations;
ablating a flat plastic plate with the ablation system per the input, the ablated plate having an ablated optical surface with high-order optical aberrations;
measuring a wavefront of the ablated optical surface;
determining a measured optical surface of the plate from the measured wavefront; and
comparing the measured optical surface to the predetermined optical surface.

2. The method of claim 1 wherein the predetermined optical surface corresponds to a plurality of predetermined expansion coefficients, wherein a plurality of the predetermined coefficients are zero.

3. The method of claim 1, further comprising identifying rotational misalignment between the measured optical surface and the predetermined optical surface.

4. The method of claim 1, further comprising identifying translational offset between the measured optical surface and the predetermined optical surface.

5. The method of claim 1 comprising adjusting the laser system to compensate for a difference between the measured optical surface to the predetermined optical surface.

6. The method of claim 10 treating a patient's eye with the adjusted laser system.

7. A closed loop system for ablating a lens, the system comprising:
a laser system having an input for a predetermined optical surface with high-order aberrations;
a flat plastic plate disposable in an optical path of a laser beam such that the laser system directs laser energy thereon in response to the predetermined optical surface so that the plate has high-order optical aberrations;
a wavefront measurement system that measures an ablated optical surface on the plate material; and
a processor configured to compare the measured ablated optical surface to the predetermined optical surface.

8. A system for testing a performance of a laser system, the system comprising:
means for ablating a surface of a plastic lens material per a predetermined optical surface having high-order aberrations;
means for measuring the ablated optical surface using a wavefront of the ablated optical surface to determine a measured optical surface of the lens material; and
means for comparing the measured optical surface to the predetermined optical surface, said means for comparing comprising means for determining at least one of translational offset between the ablated optical surface and the predetermined optical surface, or rotational offset between the measured optical surface and the predetermined optical surface.

9. A closed loop method for assessing a performance of a laser refractive surgical system, the method comprising:
choosing a set of high-order optical aberrations to determine a predetermined optical surface;
inputting the set of optical aberrations into software to direct a corneal reshaping laser system of the laser refractive surgical system to create the predetermined optical surface;
ablating a flat plate of plastic optical material with the corneal reshaping laser system of the laser refractive surgical system using the software;
measuring the ablated optical surface using an eye refractor of the laser refractive surgical system;
comparing the measured optical surface to the predetermined optical surface; and
determining at least one of:
translational offset between the ablated optical surface and the predetermined optical surface; or
rotational offset between the measured optical surface and the predetermined optical surface.
